(19) United States
(12) Patent Application Publication
(10) Pub. No.: US 2025/0288520 A1
(43) Pub. Date: Sep. 18, 2025

(54) Microemulsion Pharmaceutical Composition For Treatment Of Disorders Of The Anterior Segment Of The Eye

(71) Applicant: SIFI S.P.A., Aci Sant'Antonio (IT)

(72) Inventors: Elena SOLFATO, Mascalucia (IT); llenia ABBATE, Acireale (IT); Maria Grazia MAZZONE, Aci Sant'Antonio (IT); Francesco GIULIANO, Mascalucia (IT)

(21) Appl. No.: 18/860,409

(22) PCT Filed: Apr. 28, 2023
(86) PCT No.: PCT/EP2023/061207

§ 371 Date: Oct. 25, 2024

(30) Foreign Application Priority Data
Apr. 29, 2022 (IT) 102022000008621

(51) Int. Cl. A61K 9/107; A61K 9/00; A61K 31/44; A61K 45/06; A61K 47/10; A61K 47/14; A61K 47/26; A61K 47/44

(52) U.S. Cl. CPC A61K 9/1075; A61K 9/0051; A61K 31/44; A61K 45/06; A61K 47/10; A61K 47/14; A61K 47/26; A61K 47/44

(57) ABSTRACT

The present invention relates to an oil-in-water microemulsion that has shown to be a suitable drug depot system for the treatment of disorders of the anterior segment of the eye, wherein said disease is selected from the group consisting of corneal neovascularization disease (NV), ocular melanoma, Gelatinous drop-like corneal dystrophy (GDLD), Secondary corneal amyloidosis—GDLD type, Graves ophthalmopathy, Stevens-Johnson syndrome (SJS), Mooren's ulcer, and Corneal graft rejection and more particularly of neovascularization disease (NV). The microemulsion according to the invention is indeed capable of effectively delivering and maintaining on the anterior segment of the eye high concentrations of active ingredients after administration.

### FIELD OF THE INVENTION

The present invention relates to an oil-in-water microemulsion that has shown to be a suitable drug depot system for the treatment of disorders of the anterior segment of the eye, particularly of corneal disorders and more particularly of corneal neovascularization disease (NV). The microemulsion according to the invention is indeed capable of effectively delivering and maintaining on the anterior segment of the eye high concentrations of active ingredients after administration, thus providing an efficient drug depot system.

### BACKGROUND

The anterior segment of the eye refers to the front-most region of the eye, and includes the bulbar conjunctiva, the conjunctiva, the cornea and the sclera.

Treatments devoted to diseases of such part of the eye, need the active ingredients for ophthalmic use to be delivered and to remain in such part of the eye in effective concentrations and for a time suitable to bring their therapeutic effect.

At the same time, the barriers and drug elimination pathway of the anterior segment of the eye show unique and properties that may render difficult the possibility of maintaining a high concentration of active ingredients after administration.

In fact, it is known that eye drops for topical use are eliminated from the nasolacrimal duct two minutes after instillation, due to “eye blinking” and the different mechanisms used for elimination, and usually only 60% of the amount instilled remains on the ocular surface. (page 2, second paragraph of Clotilde J. Et al. J Control Release. 2020 May 10; 321:1-22. oi: 10.1016/j.jconrel.2020.01.057.)

In this context, for example, treatment of corneal diseases is one the main and most challenging field of research because of the peculiar properties and features of ocular surface. A healthy cornea is indeed a transparent, avascular tissue located anterior to the iris and the pupil. The cornea is unique because it is completely avascular and alymphatic, which is essential for its clarity and optimal vision. Maintaining transparency and avascularity is therefore essential to preserve optimal vision as well as protect the eye against infections and structural damage.

Abnormal new vessels can invade the corneal stroma from pre-existing pericorneal structures as a result of a disruption in the balance of angiogenic and antiangiogenic factors that normally preserve corneal transparency and subsequently lead to corneal neovascularization. This occurs due to a wide variety of ocular insults, including infection, inflammation, ischemia, degeneration, trauma, and loss of the limbal stem cell barrier.

When blood and lymphatic vessels from the pericorneal vascular plexus grow into the cornea, the result is a pathologic condition termed corneal hemangiogenesis and corneal lymphangiogenesis, respectively.

Vasculogenesis comprises the de novo formation of vessels from vascular endothelial precursor cells (i.e. hemangioblasts and angioblasts) which are derived from mesodermal precursors (via mesodermal induction).

In contrast, angiogenesis is a process in which endothelial cells of pre-existing vessels proliferate and form new vessels. In corneal neovascularization disease, the endothelial cells of newly formed corneal vessels originate from pre-existing limbal vessels (i.e. angiogenesis). However, pericytes, another crucial cell type in blood vessel formation, originate from bone-marrow derived precursors (i.e. vasculogenesis). Ozerdem and colleagues believe that both angiogenesis and vasculogenesis are involved in NV and that targeting both mechanisms would be most effective in managing this condition. [Danial Roshandel et al. Ocul Surf. 2018 October ;16(4): 398-414].

Corneal pathologies that can lead to neovascularization include lipid keratopathy, corneal ulcers and scars, herpes eye disease, infectious keratitis, chemical burns, graft rejections and hypoxic insults from contact lens wear. Corneal neovascularization is a sight threatening condition and a growing public health concern. One study reported the estimated incidence rate of 1.4 million people per year, 12% of whom suffered subsequent loss of vision. Moreover, 20% of corneal specimens taken from corneal transplant procedures have shown evidence of corneal neovascularization. [Sharif Zuhair et al. Romanian Journal of Ophthalmology, Volume 63, Issue 1, January-March 2019.]

Currently, therapeutic approach of NV involves the use of surgery and corticosteroids and NSAIDs as potent inhibitors of inflammation, cyclosporine and tacrolimus immunomodulatory agents for ocular surface immune disorders. Tocilizumab, an IL-6 receptor antagonist was effective in reducing NV in animal models by decreasing corneal inflammation and VEGF expression, TNF-α inhibitors have been used in experimental studies for their simultaneous anti-inflammatory and antiangiogenic activities, Infliximab is an anti-TNF-α monoclonal antibody.

Preclinical studies on the ANTI VEGF family have recently been conducted, but to date there is no approved therapy for NV. Among Anti VEGF agents, Aflibercept, Pegaptanib and bevacizumab have been primarily approved for use in multiple cancers for their antiangiogenic properties and are being used off-label for the treatment of ocular angiogenesis, including NV.

So far, the only topical antiangiogenic agent tested in phase II and III trials is aganirsen. It is an antisense oligonucleotide that inhibits NV via preventing insulin receptor substrate-1 (IRS-1) expression as well as downregulating expression of VEGF and IL-1 β. [Danial Roshandel et al. Ocul Surf. 2018 October ;16(4): 398-414]. Despite numerous efforts, to date there is therefore no standard procedure in the treatment of NV.

Thus, it is highly desirable to improve the current treatment for NV as well as of other diseases of the anterior segment of the eye, especially of corneal diseases.

### SUMMARY OF THE INVENTION

An object of the present invention is therefore a new and improved treatment for diseases of the anterior segment of the eye, among which especially of corneal diseases such as corneal neovascularization disease (NV). In particular, an object of the present invention is an oil-in-water microemulsion that resulted capable of effectively delivering and maintaining for an extended period of time high loadings of active ingredients for ophthalmic use on the anterior segment of the eye for the treatment of a disease, and particularly of (NV). In this way, the present invention provides an efficient drug depot system for such ophthalmic use on the anterior segment of the eye.

The Applicant has indeed surprisingly found out that oil-in-water microemulsions object of the present invention allow the absorption of active ingredients for ophthalmic use on the anterior segment of the eye and unexpectedly maintaining a high concentration of such an active after administration, even for a long time (i.e. up to 120 minutes after administration).

Hence, in a first aspect thereof, the present invention relates to an oil-in-water microemulsion for use as anterior segment ocular drug depot system in the treatment of a disease of the anterior segment of the eye, wherein said disease is selected from the group consisting of corneal neovascularization disease (NV), ocular melanoma, Gelatinous drop-like corneal dystrophy (GDLD), Secondary corneal amyloidosis—GDLD type, Graves ophthalmopathy, Stevens-Johnson syndrome (SJS), Mooren's ulcer, and Corneal graft rejection.

In preferred embodiments, the invention relates to the oil-in-water microemulsion for use as anterior segment ocular drug depot system in the treatment of preferably at least one ocular structure selected from the group consisting of: bulbar conjunctiva, conjunctiva, cornea and sclera, more preferably of the cornea, even more preferably in the treatment of corneal neovascularization disease (NV).

Notably, in a first embodiment of such aspect of the invention, the present invention relates to an oil-in-water microemulsion comprising:
an oil component in an amount of from 0.4 to 1.4% weight/volume (w/v) of the microemulsion;
an aqueous component that is in an amount of from 55.85% to 75.70% w/v of the microemulsion;
a surfactant or a mixture of a surfactant(s) and co-surfactants(s) in an amount of from 5.71 to 22.82% w/v of the microemulsion;
   for use as anterior segment ocular drug depot system in the treatment of a disease of the anterior segment of the eye, wherein said disease is selected from the group consisting of corneal neovascularization disease (NV), ocular melanoma, Gelatinous drop-like corneal dystrophy (GDLD), Secondary corneal amyloidosis—GDLD type, Graves ophthalmopathy, Stevens-Johnson syndrome (SJS), Mooren's ulcer, and Corneal graft rejection.

In this first embodiment, the oil-in-water microemulsion is used as anterior segment ocular drug depot system is in the treatment of preferably of at least one ocular structure selected from the group consisting of: bulbar conjunctiva, conjunctiva, cornea and sclera, more preferably of the cornea, even more preferably in the treatment of (NV).

Advantageously, the combination of components and their weight/volume composition allows the microemulsion according to the invention reaching a finely interspersed system composed of a lipid phase and an aqueous phase of very small particles of less than 30 nm on average, generally less than or equal to 15 nm, and a very narrow distribution of particle sizes, generally about 15±10 nm.

In a second embodiment of the first aspect thereof, the present invention relates therefore also to an oil-in-water microemulsion with average particle size of less than 30 nm, comprising:
a lipid phase comprising an oil component, one or more surfactant(s), and one or more co-surfactant(s), and
an aqueous phase comprising one or more surfactant(s), and one or more co-surfactants, wherein:
   the weight/volume ratio between the amount of surfactant(s) and co-surfactant(s) in the lipid phase to the amount of surfactant(s) and co-surfactant(s) in the aqueous phase is from 2 to 10; and
   the weight/volume ratio between the amount of oil component in the lipid phase to the amount of surfactant(s) and co-surfactant(s) in the aqueous phase is from 0.2 to 0.4; for use as anterior segment ocular drug depot system in the treatment of a disease of the anterior segment of the eye, wherein said disease is selected from the group consisting of corneal neovascularization disease (NV), ocular melanoma, Gelatinous drop-like corneal dystrophy (GDLD), Secondary corneal amyloidosis—GDLD type, Graves ophthalmopathy, Stevens-Johnson syndrome (SJS), Mooren's ulcer, and Corneal graft rejection.

In this second embodiment, the oil-in-water microemulsion is used as anterior segment ocular drug depot system is in the treatment of preferably of at least one ocular structure selected from the group consisting of: bulbar conjunctiva, conjunctiva, cornea and sclera, more preferably of the cornea, even more preferably in the treatment of corneal neovascularization disease (NV).

The advantages of the microemulsion according to this second embodiment have been disclosed in relation to the first embodiment of the first aspect of the present invention and are not herewith repeated.

Thanks to the unexpected capability of delivering to the anterior segment of the eye an active ingredient for ophthalmic use and of maintaining a high concentration on it of such active after administration, even for a long time, the oil-in-water microemulsion according to the present invention resulted particularly effective as anterior segment ocular drug depot system for the treatment of diseases of the anterior segment of the eye, wherein said disease is selected from the group consisting of corneal neovascularization disease (NV), ocular melanoma, Gelatinous drop-like corneal dystrophy (GDLD), Secondary corneal amyloidosis—GDLD type, Graves ophthalmopathy, Stevens-Johnson syndrome (SJS), Mooren's ulcer, and Corneal graft rejection., especially of corneal neovascularization disease (NV).

In a further aspect thereof, the present invention therefore relates also to a pharmaceutical composition comprising at least one active ingredient for ophthalmic use in an oil-in-water microemulsion according to the invention, for use in the treatment of diseases of the anterior segment of the eye, wherein said disease is selected from the group consisting of corneal neovascularization disease (NV), ocular melanoma, Gelatinous drop-like corneal dystrophy (GDLD), Secondary corneal amyloidosis—GDLD type, Graves ophthalmopathy, Stevens-Johnson syndrome (SJS), Mooren's ulcer, and Corneal graft rejection.

In this further aspect, the pharmaceutical composition is used in the treatment of diseases of the anterior segment of the eye in the treatment of preferably of at least one ocular structure selected from the group consisting of: bulbar conjunctiva, conjunctiva, cornea and sclera, more preferably of the cornea, even more preferably in the treatment of (NV).

The advantages of the pharmaceutical composition according to this aspect of the invention have been already disclosed with reference to the microemulsion according to the invention and are not herewith repeated.

### BRIEF DESCRIPTION OF THE DRAWINGS

In the drawings:

FIG. 1 (A-D) depicts the stability data of the recovery assay, zeta potential (pZ), average particle distribution, pH value, the polydispersity index (PDI) versus average particle distribution (size) at 25° C., at different times, up to 24 months;

FIG. 2 (A-E) depicts the stability data of the recovery assay, zeta potential (pZ), average particle distribution, pH value, the polydispersity index (PDI) and average particle distribution (size) at different temperatures 5° C., 25° C. and 40° C., at different times, up to 36 months; and

FIG. 3 (A-C) depicts the concentration of sorafenib at different time point in cornea, bulbar conjunctiva, vitreous, sclera, retina and plasma tissues after single dose administration in NZW rabbit.

### DETAILED DESCRIPTION OF THE INVENTION

In a first aspect, the present invention relates to an oil-in-water microemulsion for use as anterior segment ocular drug depot system in the treatment of a disease of the anterior segment of the eye, wherein said disease is selected from the group consisting of corneal neovascularization disease (NV), ocular melanoma, Gelatinous drop-like corneal dystrophy (GDLD), Secondary corneal amyloidosis—GDLD type, Graves ophthalmopathy, Stevens-Johnson syndrome (SJS), Mooren's ulcer, and Corneal graft rejection, especially of corneal neovascularization disease (NV).

The Applicant has surprisingly found out that oil-in-water microemulsions object of the present invention allow the absorption of active ingredients for ophthalmic use, among other sorafenib, on the anterior segment of the eye and unexpectedly maintaining a high concentration of such an active after administration, even for a long time (up to 120 minutes after administration).

Thanks to the unexpected capability of delivering to the anterior segment of the eye an active ingredient for ophthalmic use and of maintaining a high concentration on it of such active after administration, the oil-in-water microemulsion according to the invention resulted particularly effective as anterior segment ocular drug depot system for the treatment of diseases of the anterior segment of the eye, wherein said disease is selected from the group consisting of corneal neovascularization disease (NV), ocular melanoma, Gelatinous drop-like corneal dystrophy (GDLD), Secondary corneal amyloidosis—GDLD type, Graves ophthalmopathy, Stevens-Johnson syndrome (SJS), Mooren's ulcer, and Corneal graft rejection, especially of corneal neovascularization disease (NV).

An object of the present invention is therefore a new and improved treatment for diseases of the anterior segment of the eye, among which especially of corneal neovascularization disease (NV). In particular, an object of the present invention is an oil-in-water microemulsion that resulted capable of effectively delivering active ingredients for ophthalmic use, especially anti-angiogenic agents, on the anterior segment of the eye for the treatment of a disease, and particularly of the corneal neovascularization disease (NV).

Unless defined otherwise, all technical and scientific terms used herein have the same meaning as is commonly understood by one of skill in the art to which the invention(s) belong. All patents, patent applications, published applications and publications, GenBank sequences, databases, websites and other published materials referred to throughout the entire disclosure herein, unless noted otherwise, are incorporated by reference in their entirety. In the event that there is a plurality of definitions for terms herein, those in this section prevail. Where reference is made to a URL or other such identifier or address, it is understood that such identifiers can change and particular information on the internet can come and go, but equivalent information can be found by searching the internet. Reference there to evidences the availability and public dissemination of such information.

As used herein, an emulsion is a system composed of two immiscible liquid phases, intimately mixed and dispersed, the one into the other. An emulsion refers to a colloidal dispersion of two immiscible liquids, for example, an oil and water (or other aqueous liquid, e.g., a polar solvent), one of which is part of a continuous phase and the other of which is part of a dispersed phase. The emulsions herein are oil-in-water, which include any oil soluble phase dispersed in any aqueous phase, also called the water phase, in which the oil phase is the dispersed phase and the water phase is the continuous phase.

Emulsions typically are stabilized by one or more surfactants and/or emulsion stabilizers. Surfactants form an interfacial film between the oil and water phase of the emulsion, providing a metastable system.

As used herein, a microemulsion is a system composed of a lipid phase and an aqueous phase, finely interspersed. Microemulsions are liquid mixtures, transparent, isotropic and stable, of a lipid phase and an aqueous phase, held together by a surfactant, generally in conjunction with a co surfactant. Microemulsions are clear, thermodynamically stable, isotropic liquid mixtures of oil, water and surfactant, and optionally a co-surfactant.

Microemulsions form spontaneously upon mixing of the aqueous component and lipid component, optionally in presence of one or more surfactants. A microemulsion is, therefore, a thermodynamically stable system, with particles dispersed in the continuous phase. The droplet size of the dispersed phase in a microemulsion is less than 100 nm, generally in the range between 5 nm and 50 nm, inclusive.

As used herein, “surfactant” refers to synthetic and naturally occurring amphiphilic molecules that have hydrophobic portion(s) and hydrophilic portion(s). Surfactants contain a hydrophilic domain and hydrophobic domain, i.e. amphiphilic molecules. Given their nature, surfactants facilitate the formation of oil-in-water emulsions where the micelles, in order to exist, need to interact with both the water and the oil phases. Due to their amphiphilic (amphipathic) nature, surfactants and co-surfactants can reduce the surface tension between two immiscible liquids, for example, the oil and water phases in an emulsion, such as a microemulsion, stabilizing the emulsion.

Surfactants can be characterized based on their relative hydrophobicity and/or hydrophilicity. For example, relatively lipophilic surfactants are more soluble in fats, oils and waxes, typically having Hydrophobic-Lipophilic Balance (HLB) values less than 10 or about 10, while relatively hydrophilic surfactants are more soluble in aqueous compositions, for example, water, and typically have HLB values greater than 10 or about 10. Relatively amphiphilic surfactants are soluble in oil and water based liquids and typically have HLB values close to 10 or about 10. Surfactants for use in the compositions herein are biocompatible, and an HLB value between 8 or about 8 and 16 or about 16, generally 10-16, or 12-14.

As used herein, a co-surfactant is a surfactant that acts in addition to another surfactant to further reduce the surface tension of a liquid. Recitation that microemulsions contain surfactants refers to the surfactants and the co surfactants that are included. Co-surfactants are hydrophilic in nature, and reduce the surface tension of water They generally are used as wetting agents, for example, to increase the spreading abilities of a water-based fluids by reducing the surface tension of water. Cosurfactants also are used, and often needed, to increase the solubility of the primary surfactant.

As used herein, “particle size” and “average particle size” refer synonymously to the average diameter of particles in a provided liquid, for example, the droplet diameter or micelle diameter in an emulsion.

As used herein, “oil phase” or “lipid phase” is used to refer to the portion (or phase) of a composition such as those provided herein that contains one or more lipophilic ingredients and/or amphiphilic ingredients, such as an oil, and is, in general, the lipid-soluble phase. In an oil-in-water (o/w) microemulsion, the lipid phase typically is the dispersed phase while water is the dispersion phase.

As used herein, oil phase ingredient(s) refers to the components of the provided compositions that are included in the oil phase in the provided methods for making the compositions. Typical oil phase ingredients include non polar compounds, e.g., non-polar active ingredients; at least one surfactant; oils, such as non-polar solvents; preservatives; and microemulsion stabilizers. Other lipophilic and/or amphiphilic ingredients can be included in the oil phase.

As used herein, “water phase” or “aqueous phase” refers to the portion (phase) of a composition, such as those provided herein, that contains one or more hydrophilic ingredients and/or amphiphilic ingredients (water phase ingredients) and is, in general, the water-soluble phase. Typically, in the provided microemulsion compositions provided herein, the water phase is the continuous phase. “Water phase” also is used to refer to the liquid containing the water phase ingredients that is generated while preparing microemulsions. As used herein, water phase ingredient(s) refers to the components of the compositions that are included in the water phase in the provided methods for making the compositions. Typical water phase ingredients can include, but are not limited to, polar solvents, typically polar protic solvents, such as water and alcohols, typically alcohols having more than one hydroxy group such as dihydroxy and trihydroxy alcohols, such as glycerol and propylene glycol; at least one surfactant; preservatives; and emulsion stabilizers. Other hydrophilic and/or amphiphilic ingredients can be included in the water phase.

As used herein, thermodynamically stability of the microemulsions refers to the stability of the dispersion such that the phases do not separate. The microemulsions provided herein exhibit high thermodynamic stability as shown by their stability at elevated temperatures.

As used herein, a subject includes an animal, typically a mammal, typically a human. As used herein, room temperature and ambient temperature are used to describe a temperature that is common in one or more enclosed spaces in which human beings typically are or reside. Room temperature can vary, but generally refers to temperatures between 19° C. or about 19° C. and 25° C. or about 25° C. When a composition is stored at room temperature, it should be understood it is generally kept at a temperature within this range or about within this range.

As used herein, refrigerated temperature refers to a temperature that is common in a refrigerator, for example, a household or restaurant refrigerator, for example, a temperature that is cooler than room temperature, but typically a few degrees above the freezing point of water (0° C. or about 0° C., or −19° C. or −20° C.). Typically, refrigerated temperatures are between about 10° C. or about 10° C. and 0° C. or about 0° C., for example, 4° C. or about 4° C. When a composition is stored at a refrigerated temperature, it should be understood that it is kept at a temperature common to household or industrial refrigerators.

As used herein, frozen temperature refers to a temperature around or below the freezing point of water, e.g., a temperature commonly used in a household freezer, for example, 0° C. or about 0° C., for example, −19° C. or about −19° C. or −20° C. or about −20° C., or colder.

As used herein, the singular forms “a,” “an” and “the” include plural referents unless the context dictates otherwise.

As used herein, ranges and amounts can be expressed as “about” a particular value or range. About also includes the exact amount. Hence “about 5 grams” means “about 5 grams” and also “5 grams”. It also is understood that ranges expressed herein include whole numbers within the ranges and fractions thereof. For example, a range of between 5 grams and 20 grams includes whole number values such as 5, 6, 7, 8, 9, 10, 11, 12, 13, 14, 15, 16, 17, 18, 19 and 20 grams, and fractions within the range, for example, but not limited to, 5.25, 6.72, 8.5, and 11.95 grams.

As used herein, “optional” or “optionally” means that the subsequently described event or circumstance does or does not occur and that the description includes instances where said event or circumstance occurs and instances where it does not. For example, an optionally variant portion means that the portion is variant or non-variant. In another example, an optional ligation step means that the process includes a ligation step or it does not include a ligation step.

As used herein, ranges and amounts can be expressed as “about” a particular value or range. About also includes the exact amount. Hence “about 10%” means “about 10%” and also “10%.”

As used herein, “optional” or “optionally” means that the subsequently described event or circumstance or element does or does not occur, and that the description includes instances where said event or circumstance or element occurs and instances where it does not. For example, an optionally substituted group means that the group is unsubstituted or is substituted.

The present invention may present in one or more of its aspects and embodiments one or more of the characteristics disclosed hereinafter.

The oil-in water microemulsion according to the present invention comprises an oil component, an aqueous component and a surfactant or a mixture of a surfactant(s) and co-surfactants(s). Said components form a finely interspersed two-phase system composed of a lipid phase and an aqueous phase.

According to a first embodiment of the microemulsion according to the invention, an oil-in-water microemulsion is provided, comprising:
an oil component in an amount of from 0.4 to 1.4% weight/volume (w/v) of the microemulsion;
an aqueous component that is in an amount of from 55.85% to 75.70% w/v of the microemulsion;
a surfactant or a mixture of a surfactant(s) and co-surfactants(s) in an amount of from 5.71 to 22.82% w/v of the microemulsion;
   for use as anterior segment ocular drug depot system in the treatment of a disease of the anterior segment of the eye, wherein said disease is selected from the group consisting of corneal neovascularization disease (NV), ocular melanoma, Gelatinous drop-like corneal dystrophy (GDLD), Secondary corneal amyloidosis—GDLD type, Graves ophthalmopathy, Stevens-Johnson syndrome (SJS), Mooren's ulcer, and Corneal graft rejection.

In this first embodiment, the oil-in-water microemulsion is used as anterior segment ocular drug depot system in the treatment of preferably of at least one ocular structure selected from the group consisting of: bulbar conjunctiva, conjunctiva, cornea and sclera, more preferably of the cornea, even more preferably in the treatment of corneal neovascularization disease (NV).

Preferably, the oil-in-water microemulsion according to the present invention comprises from 0.4 to 1.4% weight/volume (w/v) of an oil component.

Preferably, said oil component includes at least one oil. Advantageously, said oil is biocompatible.

Said oil is preferably selected from a natural oil or synthetic oil, or mixtures thereof. Suitable natural oils, are preferably selected from vegetable and/or animal oils more preferably selected from the group consisting of: soya oil, corn oil, linseed oil, sunflower oil, krill oil, cod-liver oil, fish oil, avocado oil, almond oil, babassu oil, borage oil, carob oil, cashew nut oil, grapeseed oil, coconut oil, oryza sativa bran oil, castor oil, hemp seed oil, jojoba oil, peanut oil, poppy seed oil, sesame oil, walnut oil, olive oil, wheat-germ oil, argan oil, cottonseed oil, blackcurrant seed oil, and oils rich in PUFAs by a fraction greater than 10%.

Suitable synthetic oils are preferably selected from the group consisting of esters of medium and long-chain fatty acids, and medium and long-chain triglycerides. Preferably, said oil is a vegetable oil, for example coconut oil, or an ester of medium and long-chain fatty acid, for example isopropyl myristate.

The oil component may include also other non-polar ingredients.

Preferably, the oil-in water microemulsion according to the present invention comprises from 55.85 to 75.70% weight/volume (w/v) of an aqueous component.

Said aqueous component preferably includes water and any other polar protic solvent and cosurfactant and wetting agent optionally used in the microemulsion according to the present invention.

Preferably, the oil-in water microemulsion according to the present invention comprises from 5.71 to 22.82% weight/volume (w/v) of surfactant(s) and co-surfactants(s).

Preferably, surfactants/co-surfactants according to the present invention are non-ionic surfactants.

Preferably, the HLB of the surfactants and co-surfactants according to the present invention is from 8 to 16, such as where the HLB of each of the surfactants and co-surfactants at least 10, more preferably from 10 to 16, even more preferably from 12 to 14.

Preferably, the oil-in water microemulsion according to the present invention contains one surfactant and a co-surfactant, or two surfactants and one co-surfactant, or two surfactants and two co-surfactants.

In some embodiments, the oil-in water microemulsion according to the present invention is formed from a lipid and aqueous phase in which the surfactant is the same so that the resulting microemulsion contains one surfactant, and generally one or two co-surfactants. In other embodiments, the surfactant in each phase is different. In some embodiments, the oil-in water microemulsion according to the present invention is formed from a lipid and aqueous component in which the aqueous phase contains a surfactant and a co-surfactant, and the lipid component contains a surfactant and a co-surfactant. The co-surfactant in each phase can be the same or different.

In general, each phase of the microemulsion contains one surfactant and one co-surfactant. The surfactants and co-surfactants can be the same or different.

The skilled person readily can select surfactants and co-surfactants for use in the microemulsions. Selected surfactants will have an HLB within the requisite range and will be appropriate for the intended application and the active agent. For example, surfactants for ophthalmic use should be suitable for administration to the eye. Surfactants suitable for use in pharmaceutical compositions, include those for ophthalmic application. Such surfactants are well known (see, e.g., U.S. Pat. No. 6,267,985).

For example, in one embodiment the lipid phase contains Tween 80/propylene glycol as the surfactant/co-surfactant, and the aqueous phase contains Kolliphor RH40/propylene glycol.

Surfactants are preferably selected from the group consisting of: poloxamers, PEGylated fatty acids, polyoxyethylenes, polyoxyethylene sorbitan fatty acid derivatives, polyoxyethylenes, hydrogenated castor oil ethoxylates, glycerol esters of fatty acids, PEGylated fatty acids, polyoxyl castor oil surfactants, poloxamers, amine oxides, and alcohol ethoxylates (non-ionic). Exemplary of these are polyethylene glycol sorbitan monolaurate (polysorbate 20; TWEEN 20), polyethylene glycol sorbitan monooleate (polysorbate 80; TWEEN 80), and polyethylene glycol sorbitan monopalmitate (polysorbate 40; MONTANOX 40. In other embodiments the surfactants are selected from among polyoxyl 35 castor oil (CREMOPHOR EL, KOLLIPHOR EL), polyoxyl 40 hydrogenated castor oil (CREMOPHOR RH40; KOLLIPHOR RH 40), PEG 40 castor oil (ETOCAS 40), PEG-60 hydrogenated castor oil (CRODURET 60), and polyethylene glycol 15-hydroxystearate (KOLLIPHOR HS 15).

Preferably, the surfactant is non-ionic. For example, non-ionic surfactants can be selected from among Pluronic®, Cremophor®, Kolliphor®, Polysorbates (Tween™), lauryl dimethyl ammine oxide, polyethoxylated alcohol, polyoxyl lauryl ether, Brij®, polyoxyethylated castor oil, lecithin, poloxamers, polyethylene glycol, glycerol esters of fatty acids. Preferably, the surfactants are selected from the group consisting of: castor oil and hydrogenated castor oil ethoxylates.

Preferably, the co-surfactants include glycerol and/or propylene glycol. For example, each phase can include the same or different co-surfactant.

The oil-in water microemulsion according to the present invention optionally comprises other ingredients, such as one or more of an isotonizing (tonicity) agent, stabilizing agent, antioxidant, anti-microbial, thickening agent, and branched and linear polymers. The microemulsion according to the present invention can include for example buffers to maintain the pH at a desired range, generally between about 5.0 and 8, such as between 5.2 and 8, such as 7.4 or 7.5. Exemplary biocompatible suitable buffers include, but are not limited to: Trometamol (Tris buffer, 2-Amino-2-(hydroxymethyl) propane-1, 3-diol), Mcllvaine (citrate-phosphate buffer), Sprensen (0.133 M Na2HPO40.133 M KH2PO4 pH 7.2), sodium lactate, sodium acetate, sodium borate, boric acid, and imidazole. An exemplary buffer is histidine and citrate adjusted to pH with NaOH or HC1. The suitable buffer concentration is in the range of 10-50 mM. In the microemulsion according to the present invention, isotonizing agents may be used to achieve the required tonicity in the preparation. Exemplary of these are glycerol, sorbitol, mannitol, sucrose, trehalose, propylene glycol, dextrose, ethylene glycol, sodium chloride, potassium chloride, magnesium chloride, and calcium chloride. In one embodiment, the microemulsion comprises excipients such as: stabilizing agents, antioxidants, antimicrobials, thickening agents, branched and linear polymers. Tonicity agents are used at concentrations that guarantee osmolality of the formulation ranging, for example, between 100-400, inclusive, mOsmol/kg of the composition.

The stabilizing agent may be chosen by way of example from glycine, proline, cyclodextrins, calixarenes, hypromellose, histidine, betaine, albumin, L-carnitine, taurine, glyceryl monostearate, pectins, polyvinyl alcohol, propylene glycol. It is within the skill in the art to determine an appropriate concentration, which depends upon, for example, the particular stabilizing agent(s) and the other components of the composition. For example, the amount can be between 0.0001% to 20% w/v, inclusive. The microemulsion according to the present invention can include also thickening agents. The thickening agents include, but are not limited to, those extracted from plants, microbes and animals. Many thickening agents are known to those of skill in the art. Exemplary of thickening agents extracted from plants are gums. Exemplary of thickening agents are gums, such as gums extracted from plants belonging to the genera Cyamopsis, Sterculia, Ipomoea, Trigonella, Cassia, Physaria, Tamarindus, Ceratonia, Caesalpinia; exudates of vegetable origin, such as, by way of example those related to species belonging to the genera Manilkara, Amorphophallus, Acacia, Anogeissus, Sterculia, Astragalus; gums of microbic origin, such as, by way of example dextran, gellan gum, xanthan gum; extracts of marine origin, such as, by way of example sodium alginate, alginic acid, carrageenan, agar-agar, and derivatives of Ulva lactuca, Alga nori, Arthrospira platensis; derivatives of animal origin, such as, by way of example chitin and chitosan, hyaluronic acid; derivatives of cellulose, such as, by way of example carboxymethylcellulose (CMC), hydroxyethyl cellulose, hydroxypropyl methyl cellulose (HPMC), methylcellulose (MC), microcrystalline cellulose (MCC).

Antioxidants can also be included in the microemulsion according to the present invention. Exemplary of antioxidants are a-tocopherol, flavonoids (e.g. resveratrol, epigallocatechin-3-gallate, quercetin, naringenin, delphinidin) coenzyme Q10, NDGA (nordihydroguaiaretic acid), meso-NDGA, sodium ascorbate, L-ascorbic acid, N-acetylcarnosine, citric acid, erythorbic acid, L-6-ascorbyl palmitate, L-carnosine, L-glutathione, L-cysteine, cysteine ascorbate. The amount can be determined by the skilled person. It can depend upon the particular antioxidant(s) employed and the other components of the composition. An exemplary range is between 0.0001% and 5.0% w/v, inclusive.

Advantageously, the combination of components and their weight/volume composition allows the microemulsion according to the invention reaching a finely interspersed two-phase system composed of a lipid phase and an aqueous phase of very small particles of less than 30 nm on average, generally less than or equal to 15 nm, and a very narrow distribution of particle sizes, generally about 15±10 nm.

Advantageously, the size distribution of particles in the microemulsion is 15 nm±10 nm. Preferably, the polydispersity index (PDI) of the microemulsion is from 0.02 to 0.380, preferably from 0.02 to 0.2, more preferably from 0.02 to 0.15, more preferably of less than 0.12, even more preferably of less than 0.1.

According to a second embodiment of the first aspect thereof, the present invention relates therefore also to an oil-in-water microemulsion with average particle size of less than 30 nm, comprising:
a lipid phase comprising an oil component, one or more surfactant(s), and one or more co-surfactant(s), and
an aqueous phase comprising one or more surfactant(s), and one or more co-surfactants, wherein:
   the weight/volume ratio between the amount of surfactant(s) and co-surfactant(s) in the lipid phase to the amount of surfactant(s) and co-surfactant(s) in the aqueous phase is from 2 to 5, preferably from 2.2 to 4.7, more preferably from 2.4 to 4.5; and the weight/volume ratio between the amount of oil component in the lipid phase to the amount of surfactant(s) and co-surfactant(s) in the aqueous phase is from 0.2 to 0.4, preferably from 0.22 to 0.38, more preferably from 0.24 to 0.34;
   for use as anterior segment ocular drug depot system in the treatment of a disease of the anterior segment of the eye, wherein said disease is selected from the group consisting of corneal neovascularization disease (NV), ocular melanoma, Gelatinous drop-like corneal dystrophy (GDLD), Secondary corneal amyloidosis-GDLD type, Graves ophthalmopathy, Stevens-Johnson syndrome (SJS), Mooren's ulcer, and Corneal graft rejection.

In this second embodiment, the oil-in-water microemulsion is used as anterior segment ocular drug depot system in the treatment of preferably of at least one ocular structure selected from the group consisting of: bulbar conjunctiva, conjunctiva, cornea and sclera, more preferably of the cornea, even more preferably in the treatment of corneal neovascularization disease (NV).

The advantages, exemplary features, and preferred features of the microemulsion according to this second embodiment have been disclosed in relation to the first embodiment of the microemulsion according to present invention and are not herewith repeated.

Preferably, in the microemulsion according to this embodiment of the invention, in the lipid phase, the weight/volume ratio between the oily component and the surfactant(s) and co-surfactant(s) is from 0.05 to 0.75, more preferably from 0.07 to 0.55, even more preferably from 0.1 to 0.35.

The fine particle dispersion permits to the microemulsion according to the invention of exhibiting advantageous properties, including a resulting high thermodynamic stability. The microemulsion according to the invention may be prepared by combining particular ratios of surfactants in the aqueous and lipid (lipophilic) phases, generally, but not limited to, two surfactants (a surfactant and co-surfactants) in each phase. When the aqueous and lipophilic phases are combined, the resulting microemulsion forms. The resulting microemulsion has very small particles, less than 30 nm on average, generally less than or equal to 15 nm, and a very narrow distribution of particle sizes, generally about 15±10 nm. Among the resulting properties of the emulsions are increased thermal stability and high bioavailability of any agent formulated in the emulsions compared to similar emulsions in the prior art.

According to a preferred aspect of the invention, to achieve the advantageous properties, including the small particle size, the microemulsion according to the invention is obtainable by combining, preferably mixing, a lipid phase containing an oil component and surfactant(s) and co-surfactants(s); and an aqueous phase, also containing surfactant(s) and co-surfactants(s), wherein the weight/volume ratio between the surfactants/co-surfactants in the lipid phase and the surfactants/co-surfactants in the aqueous phase, is from 2 to 5.0, preferably from 2.2 to 4.7, more preferably from 2.4 to 4.5, and wherein the weight/volume ratio between the oil component of the lipid phase and the surfactants/co-surfactants in the aqueous phase is from 0.2 to 0.4, preferably from 0.22 to 0.38, more preferably from 0.24 to 0.34. Preferably, when the microemulsion according to the invention obtainable by the mixing process above, in the lipid phase the weight/volume ratio between the oil component and the mixture of the surfactants/co-surfactants in the lipid phase is from 0.05 to 0.75, more preferably from 0.07 to 0.55, even more preferably from 0.1 to 0.35. Preferably, when the microemulsion according to the invention obtainable by the mixing process above, the oil component is in an amount that is from 0.4 to 1.4% (w/V) of the microemulsion, the surfactant(s) and co-surfactant(s) of the lipid phase are in an amount from 4.0% to 18.67% (w/v), of the microemulsion; and the surfactant(s) and co-surfactant(s) of the aqueous phase are in amount from 1.66% to 4.15% (w/v) of the microemulsion.

Preferably, in the lipid phase the surfactant(s) comprises at least one polyoxyethylene sorbitan fatty acid derivative and at least one polyoxyl castor oil surfactant and the co-surfactant(s) comprises propylene glycol, and the weight ratio surfactant(s):co-surfactant(s) is of 3:1.5, in which preferably in the surfactant(s) the ratio polyoxyethylene sorbitan fatty acid derivative: polyoxyl castor oil surfactant is of 1:2. Preferably, in the aqueous phase the surfactant(s) comprises at least one polyoxyl castor oil surfactant and the co-surfactant(s) comprises propylene glycol, and the ratio surfactant(s):co-surfactant(s) is of 3:1.

Preferably, the polyoxyethylene sorbitan fatty acid derivative is selected from polyethylene glycol sorbitan monolaurate (polysorbate 20; TWEEN 20), polyethylene glycol sorbitan monooleate (polysorbate 80; TWEEN 80), and polyethylene glycol sorbitan monopalmitate (polysorbate 40; MONTANOX 40), more preferably is polyethylene glycol sorbitan monooleate.

Preferably polyoxyl castor oil surfactant is selected from polyoxyl 35 castor oil (CREMOPHOR EL, KOLLIPHOR EL), polyoxyl 40 hydrogenated castor oil (CREMOPHOR RH40; KOLLIPHOR RH 40), PEG 40 castor oil (ETOCAS 40), PEG-60 hydrogenated castor oil (CRODURET 60), and polyethylene glycol 15-hydroxystearate (KOLLIPHOR HS 15), more preferably is polyoxyl 40 hydrogenated castor oil.

In a preferred embodiment, the microemulsion according to the invention obtainable by the process above, is obtainable by the following steps of:
a) preparing a lipid phase, where a mixture of surfactants/co-surfactants is solubilized in an oil, mixing from 1 to 10 parts of the mixture for each oil part;
b) preparing an aqueous phase, where one or more of a surfactant and/or co-surfactants is solubilized in water;
c) titrating the lipid phase with the aqueous phase to obtain microemulsion taking into account the titration rate is preferably described by the following equation:

Vtit=k×(vtot/1000 milliliters);

where Vtit=titration speed in ml×min−1, k=2 ml×min−1, and Vtot=total volume of the formulation in ml;
where: 0.4-1.4% (w/v) of the microemulsion is composed of the oil component of the lipid phase; 4.0%-18.67% (w/v) of the microemulsion is composed of the surfactants/co surfactants of the lipid phase; and 1.66%-4.15% (w/v) of the total surfactants/co-surfactants are the surfactants/co-surfactants of the aqueous phase. In another embodiment, the microemulsion according to the invention obtainable by the process above, is obtainable by the following steps of:
a) preparing a lipid phase, which optionally includes a pharmaceutically active agent if a pharmaceutical composition according to the invention is prepared, and in which a mixture of surfactants/co-surfactants is solubilized in oil, mixing from 1 to 10 parts of the mixture for each oil part;
b) preparing a aqueous phase, where one or more of a surfactant and/or co-surfactants is solubilized in water;
c) titrating the lipid phase with the aqueous phase to obtain a microemulsion, where: 0.4-1.4% (w/v) of the microemulsion is composed of the oily component of the lipid phase; 4.0%-18.67% (w/v) of the microemulsion is composed of the surfactants/ co-surfactants of the lipid phase, and 1.66%-4.15% (w/v) of the microemulsion is composed of surfactants/co-surfactants of the aqueous phase.

If a pharmaceutical composition according to the invention is prepared, the pharmaceutically active agent is preferably included in an amount of from 0.001 mg/ml to 50 mg/ml, more preferably from 0.01 mg/l to 50 mg/ml, even more preferably 0.01 mg/ml to 30 mg/ml of the composition.

Pharmaceutically active agents included or added to the compositions so prepared include any suitable for treatment of a particular disorder, including any listed herein above and below.

In accord with these methods, the titration rate is preferably described by the following equation:

Vtit=k×(vtot/1000 milliliters);

where Vtit=titration speed in ml×min−1, k=2 ml×min−1, and Vtot=total volume of the formulation in ml.

The resulting microemulsion and compositions advantageously exhibit dispersed particles with a fine dispersion, preferably with an average size ≤30 nm, and preferably with a size distribution of particles of 15 nm±10 nm, and/or a polydispersity index (PDI) of less than or equal to 0.380, more preferably in the range from 0.01 to 0.380, more preferably from 0.01 to 0.20, more preferably from 0.01 to 0.15, more preferably less than 0.2, even more preferably less than 0.1.

In other methods, the microemulsion and composition according to the invention is obtainable by a method comprising the steps of:
a) preparing a lipid phase comprising an oil component, one or more surfactant(s) and one or more co-surfactant(s), and optionally a pharmaceutically active agent if a pharmaceutical composition according to the invention is prepared, and
b) an aqueous phase comprising one or more surfactant(s) and one or more co-surfactant(s),
   wherein the weight/volume ratio between the amount of surfactant(s) and co-surfactant(s) in the lipid phase to the amount of surfactant(s) and co-surfactant(s) in the aqueous phase is from 2 to 5 and wherein the weight/volume ratio between the amount of oil component in the lipid phase to the amount of surfactant(s) and co-surfactant(s) in the aqueous phase is from 0.2 to 0.4; and, then
c) combining lipid phase with the aqueous phase.

In such a way, an oil-in-water microemulsion and a composition according to the invention is formed, in which preferably the average particle size is less than 30 nm. Step c) is preferably effected by titrating the lipid phase with the aqueous phase, where the titration rate is described by the following equation:

Vtit=k×(vtot/1000 milliliters);

where
Vtit is the speed in ml×min−1, k=2 ml×min−1, and Vtot=total volume of the formulation in ml.

In these methods, the weight/volume ratio between the amount of surfactant and co-surfactant in the lipid phase to the amount of surfactant and co-surfactant in the aqueous phase is preferably of from 2.2 to 4.7, more preferably of from 2.4 to 4.5. The microemulsion according to the invention provided herein is to be used as pharmaceutically acceptable depot system for formulating active ingredient for ophthalmic use to the anterior segment of the eye, preferably to one ocular structure selected from the group consisting of: bulbar conjunctiva, conjunctiva, cornea and sclera, more preferably to the cornea. The microemulsion provided is used to formulate such active agents in a form that is stable and can also be preserved at room temperature.

Thanks to the unexpected capability of delivering to the anterior segment of the eye an active ingredient for ophthalmic use and of maintaining a high concentration on the same of such active after administration, even for a long time, the oil-in-water microemulsion according to the present invention resulted particularly effective as anterior segment ocular drug depot system for the treatment of diseases the anterior segment of the eye, preferably of at least one ocular structure selected from the group consisting of: bulbar conjunctiva, conjunctiva, cornea and sclera, more preferably of the cornea, even more preferably for the treatment of corneal neovascularization disease (NV).

In a further aspect thereof, the present invention therefore relates also to a pharmaceutical composition comprising at least one active ingredient for ophthalmic use in an oil-in-water microemulsion according to the invention, for use in the treatment of diseases of the anterior segment of the eye, preferably of at least one ocular structure selected from the group consisting of: bulbar conjunctiva, conjunctiva, cornea and sclera, more preferably of the cornea, even more preferably in the treatment of corneal neovascularization disease (NV).

Notably, in a first embodiment of this further aspect thereof, the present invention relates therefore to a pharmaceutical composition comprising at least one active ingredient for ophthalmic use in an oil-in-water microemulsion comprising:
an oil component in an amount of from 0.4 to 1.4% weight/volume (w/v) of the microemulsion;
an aqueous component that is in an amount of from 55.85% to 75.70% w/v of the microemulsion;
a surfactant or a mixture of a surfactant(s) and co-surfactants(s) in an amount of from 5.71 to 22.82% w/v of the microemulsion;
   for use in the treatment of diseases of the anterior segment of the eye, wherein said disease is selected from the group consisting of corneal neovascularization disease (NV), ocular melanoma, Gelatinous drop-like corneal dystrophy (GDLD), Secondary corneal amyloidosis—GDLD type, Graves ophthalmopathy, Stevens-Johnson syndrome (SJS), Mooren's ulcer, and Corneal graft rejection.

In this first embodiment of the further aspect, the pharmaceutical composition is used in the treatment of diseases of the anterior segment of the eye in the treatment of preferably of at least one ocular structure selected from the group consisting of: bulbar conjunctiva, conjunctiva, cornea and sclera, more preferably of the cornea, even more preferably in the treatment of corneal neovascularization disease (NV).

In a second embodiment of this further aspect thereof, the present invention relates therefore to also to a pharmaceutical composition comprising at least one active ingredient for ophthalmic use in an oil-in-water microemulsion with average particle size of less than 30 nm, comprising:
a lipid phase comprising an oil component, one or more surfactant(s), and one or more co-surfactant(s), and
an aqueous phase comprising one or more surfactant(s), and one or more co-surfactants, wherein:
   the weight/volume ratio between the amount of surfactant(s) and co-surfactant(s) in the lipid phase to the amount of surfactant(s) and co-surfactant(s) in the aqueous phase is from 2 to 5, preferably from 2.2 to 4.7, more preferably from 2.4 to 4.5; and the weight/volume ratio between the amount of oil component in the lipid phase to the amount of surfactant(s) and co-surfactant(s) in the aqueous phase is from 0.2 to 0.4, preferably from 0.22 to 0.38, more preferably from 0.24 to 0.34;
   for use in the treatment of diseases of the anterior segment of the eye, wherein said disease is selected from the group consisting of corneal neovascularization disease (NV), ocular melanoma, Gelatinous drop-like corneal dystrophy (GDLD), Secondary corneal amyloidosis—GDLD type, Graves ophthalmopathy, Stevens-Johnson syndrome (SJS), Mooren's ulcer, and Corneal graft rejection.

In this second embodiment of the further aspect, the pharmaceutical composition is used in the treatment of diseases of the anterior segment of the eye in the treatment of preferably of at least one ocular structure selected from the group consisting of: bulbar conjunctiva, conjunctiva, cornea and sclera, more preferably of the cornea, even more preferably in the treatment of NV.

The advantages of the pharmaceutical composition according to this aspect of the invention have been already disclosed with reference to the microemulsion according to the invention and are not herewith repeated.

With regard to the pharmaceutical composition according to the invention, the oil-in-water microemulsion preferably shows the advantages, exemplary features, and preferred features of the microemulsion disclosed above in the present application. In the pharmaceutical composition, the active ingredient for ophthalmic use is preferably selected from the group consisting of: an anti-angiogenic agent or Multikinase inhibitors agent, immunosuppressant and immunomodulating agent, anti-oxidant agent, and anti-inflammatory agent.

Preferably, said anti-angiogenic agent or Multikinase inhibitors agent is selected from the group consisting of: sorafenib, sorafenib tosylate, regorafenib, regorafenib tosylate, regorafenib isethionate, regorafenib, ethylsulfonate apremilast, radotinib, spironolactone, and pharmaceutically acceptable derivatives, hydrates, solvates, metabolites and salts thereof, or a polymorphic crystalline form thereof.

Preferably, said anti-oxidant agent is selected from the group consisting of: nordihydroguaiaretic acid, meso-nordihydroguaiaretic (masoprocol) and pharmaceutically acceptable derivatives, hydrates, solvates, metabolites and salts thereof, or a polymorphic crystalline form thereof.

Preferably, said anti-inflammatory agent is selected from the group consisting of: Non-Steroidal Anti-Inflammatory Drugs (NSAIDs) preferably selected from the group consisting of: Diclofenac, Diflunisal, Etodolac, Fenoprofen, Flurbiprofen, Ibuprofen, Indomethacin, Ketoprofen, Ketorolac, Mefenamic acid, Meloxicam, Nabumetone, Naproxen, Oxaprozin, Piroxicam, Sulindac, Tolmetin, COX-2 Selective NSAIDs, Celecoxib, Rofecoxib, and Valdecoxib, and corticosteroid anti-inflammatory drugs preferably selected from the group consisting of bethamethasone, prednisone, prednisolone, triamcinolone, methylprednisolone, dexamethasone and hydrocortisone, cortisone, ethamethasoneb, and pharmaceutically acceptable derivatives, hydrates, solvates, metabolites and salts thereof, or a polymorphic crystalline form thereof.

Preferably, said immunosuppressant and immunomodulating agent is selected from the group consisting of: interleukin inhibitors, preferably apremilast, Daclizuma, Natalizumab, tocilizumab, mepolizumab, ixekizumab, secukinumab, reslizumab and pharmaceutically acceptable derivatives, hydrates, solvates, metabolites and salts thereof, or a polymorphic crystalline form thereof, and TNF alfa inhibitors, preferably certolizumab, etanercept, adalimumab, golimumab, infliximab, and pharmaceutically acceptable derivatives, hydrates, solvates, metabolites and salts thereof, or a polymorphic crystalline form thereof.

In a preferred embodiment, in the pharmaceutical composition according to the invention said at least one active ingredient for ophthalmic use is an anti-angiogenic agent, more preferably selected from the group consisting of: sorafenib, sorafenib tosylate, and pharmaceutically acceptable derivatives, hydrates, solvates, metabolites and salts thereof, or a polymorphic crystalline form thereof.

In the pharmaceutical composition, the amount of the active ingredient for ophthalmic use may vary upon the active ingredient itself and indication, posology, dosage regimen for which it is intended.

In a preferred embodiment, the pharmaceutical composition comprises from 0.001 mg/ml to 50 mg/ml, more preferably from 0.01 mg/ml to 50 mg/ml, even more preferably from 0.01 mg/ml to 30 mg/ml of at least one active ingredient for ophthalmic use.

The microemulsion and pharmaceutical composition according to the invention can be used for topical and local application. Preferably, the microemulsion and pharmaceutical composition according to the invention are formulated for topical application, such as eye drops.

The microemulsion and pharmaceutical composition according to the invention can be used in the treatment of several diseases of the anterior segment of the eye.

Preferably, the anterior segment of the eye is at least one ocular structure selected from the group consisting of: bulbar conjunctiva, conjunctiva, cornea and sclera, more preferably the cornea.

The disease to be treated with the microemulsion and pharmaceutical composition according to the invention is selected from the group consisting of: a disease of the bulbar conjunctiva, a disease of the conjunctiva, a corneal disease, a disease of sclera, more preferably a corneal disease.

Diseases that can be treated with microemulsion and pharmaceutical composition according to the invention are selected from the group consisting of: corneal neovascularization disease (NV), ocular melanoma,, in particular Uveal melanoma and conjunctival melanoma, Gelatinous drop-like corneal dystrophy (GDLD), Secondary corneal amyloidosis—GDLD type, Graves ophthalmopathy, Stevens Johnson syndrome (SJS), Mooren's ulcer and Corneal graft rejection.

Ocular melanoma is the second most common type of melanoma after cutaneous. It arises from melanocytes situated in conjunctival membrane and uveal tract of the eye. Although rarely, it can also arise from melanocytes located in the orbit. Uvea is the most frequent site of origin of ocular melanomas and comprises 82.5% of all of them, while conjunctival melanoma is far less common. Great majority of ocular melanomas are primary however, metastatic melanoma from primary cutaneous site can also occur in the ocular region, and it accounts for less than 5% of all metastases to the eye and orbit.

Iris or anterior uveal melanoma is the least common uveal melanoma, but has more benign clinical course compared with posterior uveal melanoma. Most patients with uveal melanoma are age between 50 and 80 years, with peak in seventies, and mean age at diagnosis 58 years. Iris melanoma is more common among young patients, respectively.

Conjunctival melanomas arise from melanocytes located in the basal layer of the epithelium of the conjunctival membrane. Unlike the other mucous membranes, bulbar part of the conjunctiva is directly exposed to sun radiation. Conjunctival melanoma is very rare and comprises about 5% of all melanomas in the ocular region. Conjunctival melanoma almost exclusively occurs in whites, and only less than 1% are African-American patients. It does not show a predilection for either gender. Incidence of conjunctival melanoma is increasing with age; more than half patients are age over 60 years (54%), while it is extremely rare in younger than 20 years (1%).

Gelatinous drop-like corneal dystrophy (GDLD) is an autosomal recessive disease characterised by corneal subepithelial and stromal deposits of gelatinous amyloid material, insidious in onset and presents usually in the first two decades of life. It was first described by Nakaizumi1 in 1914 and named as gelatinous drop-like dystrophy, prior to which, it was known as primary familial amyloidosis or subepithelial amyloidosis. It is bilaterally symmetric, has low degree of penetrance and manifests commonly between 8 and 18 years of age. 2 3 GDLD is a monogenic disease caused due to a homozygous biallelic loss-of-functional mutation of the tumour-associated calcium signal transducer 2 (TACSTD2) gene, located on the short-arm of chromosome 1.4 This gene encodes for TACSTD2 protein which is implicated in the disease pathogenesis.

Secondary corneal amyloidosis—GDLD type is known to occur after chronic ocular irritation/inflammatory/infective disorders like trichiasis, entropion, keratoconus and so forth. It has been classified into three subtypes according to their clinical appearance. The GDLD type secondary corneal amyloidosis resembles GDLD lesions morphologically, having milky white soft masses on the corneal surface. Correlation with clinical history is of paramount importance for its differentiation from primary corneal amyloidosis. The deposits seem to be confined to the site of inflammation or chronic irritation

Graves ophthalmopathy (also known as Graves' disease) is an autoimmune disease caused by antibodies directed against receptors present in the thyroid cells and also on the surface of the cells behind the eyes. Rarely can also affect the skin, usually the front part of the legs. This usually results in a generalized over activity of the thyroid gland (hyperthyroidism). Up to one-half of people with Graves' disease develop eye symptoms. The most common clinical features of Graves' ophthalmopathy are upper eyelid retraction, edema, and erythema of the periorbital tissues and conjunctivae, and proptosis. Approximately 3 to 5% of patients with Graves' ophthalmopathy have severe disease with intense pain, inflammation, and sight-threatening corneal ulceration or compressive optic neuropathy. [Rebecca S. Bahn et al. N Engl J Med. 2010 Feb. 25; 362(8): 726-738]

Stevens-Johnson syndrome (SJS) is a toxic epidermal necrolysis (TEN) represent different ends of the spectrum of the same clinical entity causing severe mucocutaneous reactions, usually to drugs, characterized by intraepidermal cell death leading to blistering and epidermal sloughing. The severe cutaneous manifestations of this disease spectrum may often lead to overlooking of the ocular sequelae, which are very common and may lead to loss of visual acuity. The lid complications included discharge, lid margin ulceration and crusting, lid edema, matting of eyelashes, meibomitis, blepharitis, dystrichiasis, and peeling of skin over lids. Conjunctival complications included conjunctivitis, conjunctival membranes, subconjunctival hemorrhage, and symblepharon. Corneal complications included superficial punctate epithelial keratitis, corneal ulceration, and punctate epithelial erosions. [Abrol, et al.: Ocular manifestations of SJS/TEN, Indian Dermatology Online Journal |Volume 11| Issue 4|July-August 2020]

Mooren's ulcer is a rapidly progressive, painful, ulcerative keratitis which initially affects the peripheral cornea and may spread circumferentially and then centrally. Mooren's ulcer can only be diagnosed in the absence of an infectious or systemic cause and must be differentiated from other corneal abnormalities, such as Terrien's degeneration. Although the etiology remains unknown, recent research has proposed an underlying immune process and a possible association with the hepatitis C virus. The response to medical and surgical intervention is typically poor, and the visual outcome can be devastating. These ulcers usually respond poorly to conventional therapy, as there is limited knowledge of the pathophysiology of the disease. Evidence of an autoimmune component advocates the use of steroids and immunosuppressive agents.

Corneal graft rejection: penetrating keratoplasty is the most widely practiced type of transplantation in humans. Irreversible immune rejection of the transplanted cornea is the major cause of human allograft failure in the intermediate and late postoperative period. This immunological process causes reversible or irreversible damage to the grafted cornea in several cases despite the use of intensive immunosuppressive therapy. Corneal graft rejection comprises a sequence of complex immune responses that involves the recognition of the foreign histocompatibility antigens of the corneal graft by the host's immune system, leading to the initiation of the immune response cascade. An efferent immune response is mounted by the host immune system against these foreign antigens culminating in rejection and graft decompensation in irreversible cases. A variety of donor-and host-related risk factors contribute to the corneal rejection episode. Epithelial rejection, chronic stromal rejection, hyperacute rejection, and endothelial rejection constitute the several different types of corneal graft rejection that might occur in isolation or in conjunction. Corneal graft failure subsequent to graft rejection remains an important cause of blindness and hence the need for developing new strategies for suppressing graft rejection is colossal [Anita Panda eta al. Surv Ophthalmol 52 (4) July-August 2007]

In a preferred embodiment, the microemulsion and pharmaceutical composition according to the invention are used for the treatment of corneal neovascularization disease (NV).

Further features and advantages of the invention will appear more clearly from the following description of some preferred embodiments thereof, made hereinafter by way of a non-limiting example with reference to the following exemplary examples.

### EXPERIMENTAL PART

### Example 1: Illustrative Microemulsions

Two formulations were obtained using sorafenib tosylate as active ingredient, isopropyl myristate as an oil phase, water, Tween 80 and Kolliphor RH40 as emulsifying agents (surfactants), propylene glycol (co-surfactants) as a wetting agent, sodium citrate dihydrate as a buffer, and citric acid as a pH adjuster. As detailed below, the emulsifiers and the wetting agent were the surfactant and co-surfactants, respectively, in the lipid phase and/or in the aqueous phase, respectively.

Microemulsion formulation 2-F1:

| Components | % w/v |
|---|---|
| Sorafenib tosylate | 0.300 |
| Isopropyl myristate | 1.410 |
| Tween 80 | 4.150 |
| Kolliphor RH40 | 11.410 |
| Propylene glycol | 7.260 |
| Sodium citrate dihydrate | 0.500 |
| Purified water | q.s. 100 ml |

Formulation 2-F1 was prepared using Isopropyl myristate, as an oil phase component, water as a dispersant, Tween 80, Propylene glycol and Kolliphor RH40 were the surfactants/co-surfactants of the lipid phase. In the lipid phase, the Tween 80, the propylene glycol and the Kolliphor RH40 were in a ratio of 1:2:1.5. Kolliphor RH40 and propylene glycol were the surfactants/co-surfactants in the aqueous phase, where, in the aqueous phase, the Kolliphor RH40 and the propylene glycol were in a ratio of 3:1. In this formulation, the surfactants/co-surfactants of the lipid phase were 18.67% (w/v) of the microemulsion and the surfactants/co-surfactants of the aqueous phase were 4.15% (w/v) of the microemulsion.

Microemulsion formulation 2-F2:

| Components | % w/v |
|---|---|
| Sorafenib tosylate | 0.300 |
| Coconut oil | 1.410 |
| Tween 80 | 4.150 |
| Kolliphor RH40 | 11.410 |
| Propylene glycol | 7.260 |
| Sodium citrate dihydrate | 0.500 |
| Purified water | q.s. 100 ml |

Formulation 2-F2 was prepared using coconut oil, as an oil phase component, water as a dispersant, Tween 80 and Kolliphor RH40 as emulsifying agents, propylene glycol as a wetting agent, sodium citrate dihydrate as a buffer and citric acid as a pH adjuster. Tween 80, propylene glycol and Kolliphor RH40 were the surfactants/co-surfactants of the lipid phase, where, in the lipid phase, the Tween 80, the propylene glycol and the Kolliphor RH40 were in a ratio of 1:2:1.5; Kolliphor RH40 and propylene glycol were the surfactants/co-surfactants of the aqueous phase, where, in the aqueous phase, the Kolliphor RH40 and the propylene glycol were in a ratio of 3:1. In this formulation, the surfactants/co-surfactants of the lipid phase were 4.05% (w/v) of the microemulsion and the surfactants/co-surfactants of the aqueous phase were 1.66% (w/v) of the microemulsion.

The chemical physical parameter that characterizes the formulations are reported in table 1 and 2

TABLE 1
Chemical physical characterization formulation 2-F1

| Test | Unit | Range |
|---|---|---|
| Appearance | — | clear colourless solution |
| Z potential | mV | −15.0-15.0 |
| Recovery | % | 95-105 |
| pH | — | 6.0-7.0 |
| Particle size | nm | 15.0 ± 10.0 nm |
| Polydispersity index | — | ≤0.2 |

TABLE 2
Chemical physical characterization formulation 2-F2

| Test | Unit | Range |
|---|---|---|
| Appearance | — | clear colourless solution |
| Z potential | mV | −15.0-15.0 |
| Recovery | % | 95-105 |
| pH | — | 6.0-7.0 |
| Particle size | nm | 15.0 ± 10.0 nm |
| Polydispersity index | — | ≤0.34 |

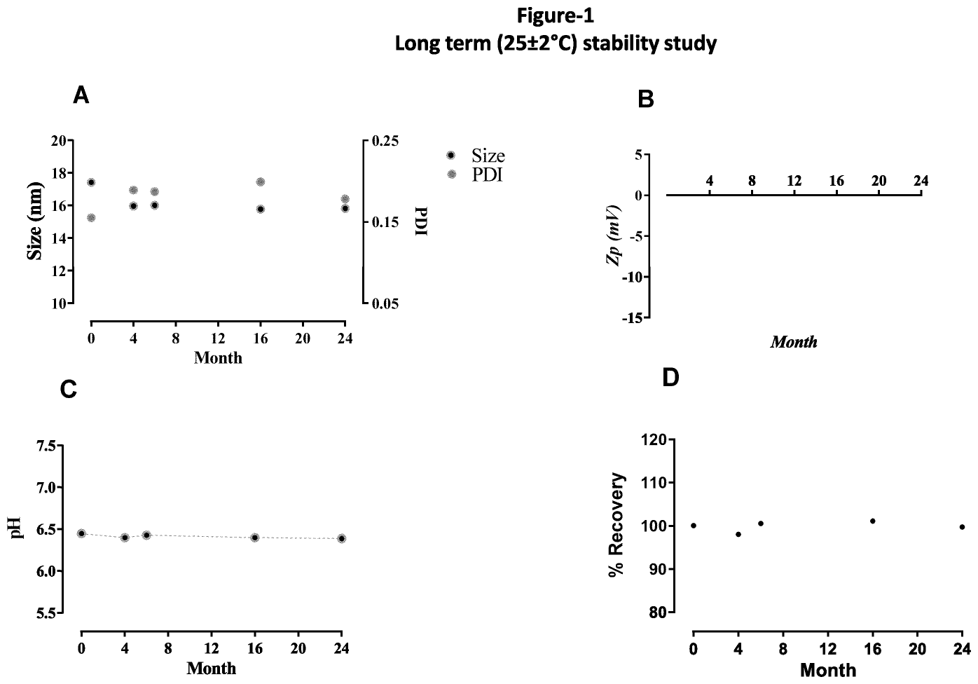

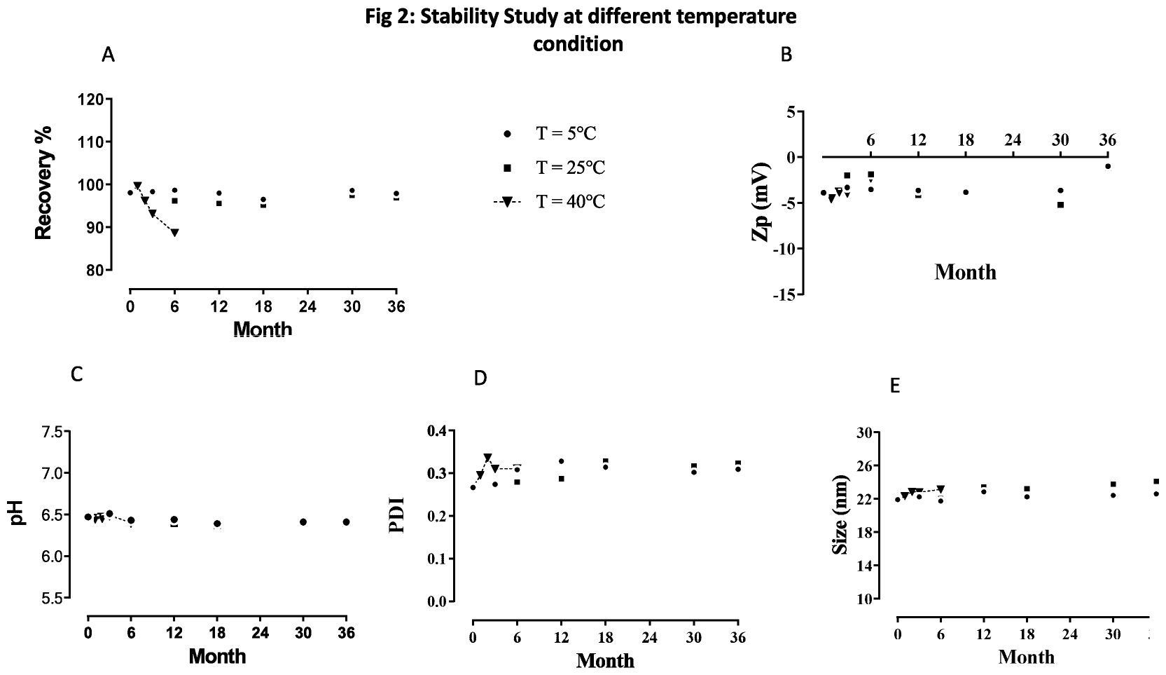

The stability data in long term condition at 25° C. highlighted a strong stability for formulation 2-F1 for all chemical physical parameter considered (FIG. 1, A-D). The recovery assay of sorafenib is inside specification range proposed for all period under study (FIG. 1 A). The polydispersity index (PDI) versus average particle distribution (size) show a very low size of the droplets (≤20 nm) and a very low and homogenous polydispersity index (≤0.20).
The stability data for formulation 2-F2 (FIG. 2 A-E) highlighted a strong stability profile for all chemical physical parameter considered (FIG. 2, A-E) in all condition tested (5° C., 25° C. and 40° C.). The recovery assay of sorafenib is inside specification range proposed for all period under study in long term condition (FIG. 2A). The polydispersity index (FIG. 2 D) exhibits a very low and homogenous value (PDI ≤0.34) and the droplets show a low average particle distribution (size ≤22 nm).

### Example 2: Bioavailability Study After Single Dose Administration of Sorafenib Microemulsion (Formulation 2-F1)

Bioavailability study in NZW rabbits after single dose administration (50 μL topical administration in the right eye) was conducted and content of sorafenib tosylate in plasma (PL), bulbar conjunctivae (BCJ), aqueous humor (AH), cornea (C), vitreous (V), retina (R), and sclera (SC) was evaluated after treatment at t=5, 15, 30, 60, 90, 120 min by LC-MS.

Thirty-six NZW male rabbits, weighing approximately 2 kg, were used in this study. The study was conducted in accordance with GLP standards at the test facility IRIS PHARMA. All animals were treated according to the Directive 2010/63/UE European Convention for the Protection of Vertebrate Animals used for Experimental and Other Scientific Purposes and to the ARVO Statement for the Use of Animals in Ophthalmic and Vision Research. All animals were housed individually in standard cages under identical temperature (18° C.±3° C.), relative humidity (45%-80%), and controlled enrichment conditions and exposed to a 12-h light and darkness cycle in continuously ventilated rooms (15-20 air volumes per hour). They received a standard dry pellet diet and water ad libitum. Only animals with no macroscopic ocular signs of irritation were involved in this study and randomly divided in six time-point groups of 6 animals each (Table 3).

TABLE 3
Study design

| Group | | Dose | Time-points | | |
|---|---|---|---|---|---|
| no | Treatment | regimen | post dose | | Animal number |
| 1 | NaMESys- | Single 50-μL | 5 | min | 1, 2, 3, 4, 5, 6 |
| 2 | SOR | instillation | 15 | min | 7, 8, 9, 10, 11, |
| | | in right eye | | | 12 |
| 3 | | | 30 | min | 13, 14, 15, 16, |
| | | | | | 17, 18 |
| 4 | | | 1 | h | 19, 20, 21, 22, |
| | | | | | 23, 24 |
| 5 | | | 1.5 | h | 25, 26, 27, 28, |
| | | | | | 29, 30 |
| 6 | | | 2 | h | 31, 32, 33, 34, |
| | | | | | 35, 36 |

Animals received a single 50 μL topical administration in the right eye and at the time-points listed in Table 3 they were anesthetized by an intramuscular injection of a mix xylazine/ketamine before euthanasia by an intracardiac injection of overdosed pentobarbital. The whole blood in EDTA anticoagulant tubes were sampled by intracardiac puncture, and plasma was prepared and frozen at −23° C.±7° C. Immediately after euthanasia, BCJ, AH, C, V, R, and SC were quickly and carefully dissected from both eyes and weighed, frozen in dry ice and at −23° C.±7° C. until assay. Content of sorafenib was determined in R and PL of both eyes in priority and in AH, BCJ, C, V and SC of both eyes following the rapid resolution liquid chromatography/tandem mass spectrometry method.

Under the experimental conditions described and after a single 50 μL instillation of NaMESys-SOR in right eye of albino rabbits it can be stated that:
in retina of treated eyes, sorafenib maximum content (170 ng/g) was observed 5 min after treatment and decreased rapidly 2 h post dose. Sorafenib was not quantified in left untreated eyes for all rabbits:
sorafenib was not quantified in plasma for all rabbits 5 min to 30 min post dose and 5/6 rabbits, 2/6 rabbits and 1/6 rabbits, 1 h, 1.5 h and 2 h post dose, respectively. Sorafenib maximum content (1.7 ng/ml) was observed 2 h after treatment;
sorafenib was quantified in bulbar conjunctivae, cornea and sclera for all rabbits in treated eye and not quantified in untreated eye (except in bulbar conjunctivae 2/36 rabbits, cornea 3/36 rabbits and in sclera 3/36 rabbits). Sorafenib quantified in left eyes for bulbar conjunctivae, cornea and sclera can be linked to the contamination by the animal (rubbing eyes with his paws). Sorafenib maximum content was observed 5 min after treatment for bulbar conjunctivae and sclera and 15 min after treatment in cornea and slowly decreases until 2 h post dose;
in vitreous humor of treated eyes, sorafenib maximum content was observed 5 min after treatment and decreased rapidly 30 min post dose. Sorafenib was not quantified in untreated eyes and for some rabbits treated eyes (18/36).

In the Biodistribution study, the diffusion of the drug was affected by many factors:
Molecular weight of the drug;
Partition coefficient of the drug;
size and charge of drug depot system;
lipophilicity of the membrane to cross.

Smaller and more lipophilic drugs are mainly cleared in the posterior segment of the eye due to their ability of crossing the blood-retinal barrier (BRB).

Positively charged nanoparticles seem to present more difficulty than neutral and negatively charged ones to cross membrane barrier.

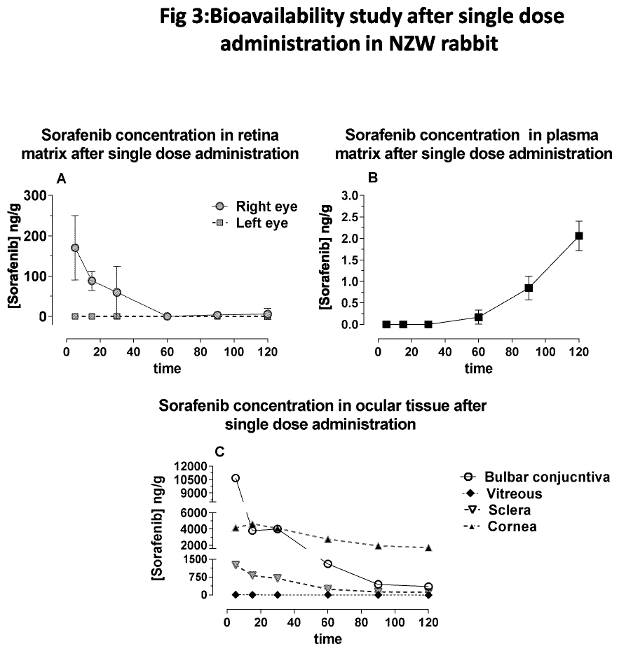

Microemulsion described in present application was composed of an appropriate Hydrophilic/Lipophilic balance due to ratio of lipids phase and aqueous phase and was characterized by negatively charged particle and showed ability to cross blood-retinal barrier (BRB) as demonstrated by data show (FIG. 3 A), where sorafenib maximum content in retina of treated eyes was observed 5 min after treatment and it was equal to 170 ng/g. In addition, as it was evident no amount of sorafenib was detected in the left untreated eye for these tissues. The highest detected amount of sorafenib in plasma was found two hours after treatment, and it was equal to 2 ng/g. This ruled out the possibility of a systemic depot of sorafenib (FIG. 3B).

Surprisingly, the study showed a huge amount of sorafenib in cornea, bulbar conjunctiva and sclera (FIG. 3C). In particular the study highlighted an high concentration of sorafenib in cornea after 120 min (2 h) post treatment (1712 ng/g SD=±596), while in other tissue (sclera and vitreous) the concentration of sorafenib decreased after 30 min post treatment.

It can be concluded that the new drug depot system stabilized and made sorafenib in liquid formulation more bioavailable for ocular surface as if a reserve effect was promoted; the depot system loaded with sorafenib demonstrated to be a tool to improve pharmacotherapy in Corneal Neovascularization disease.

1. A method for the treatment of a disease of the anterior segment of the eye comprising the step of using as anterior segment ocular drug depot system an oil-in-water microemulsion comprising:
an oil component in an amount of from 0.4 to 1.4% weight/volume (w/v) of the microemulsion;
an aqueous component that is in an amount of from 55.85% to 75.70% w/v of the microemulsion;
a surfactant or a mixture of a surfactant(s) and co-surfactants(s) in an amount of from 5.71 to 22.82% w/v of the microemulsion;
wherein said disease is selected from the group consisting of: corneal neovascularization disease (NV), ocular melanoma, Gelatinous drop-like corneal dystrophy (GOLD), Secondary corneal amyloidosis—GDLD type, Graves ophthalmopathy, Stevens-Johnson syndrome (SJS), Mooren's ulcer, and Corneal graft rejection.

2. A method for the treatment of a disease of the anterior segment of the eye comprising the step of using as anterior segment ocular drug depot system an oil-in-water microemulsion with average particle size of less than 30 nm, comprising:
a lipid phase comprising an oil component, one or more surfactant(s), and one or more co-surfactant(s), and
an aqueous phase comprising one or more surfactant(s), and one or more co-surfactants, wherein:
the weight/volume ratio between the amount of surfactant(s) and co-surfactant(s) in the lipid phase to the amount of surfactant(s) and co-surfactant(s) in the aqueous phase is from 2 to 5; and the weight/volume ratio between the amount of oil component in the lipid phase to the amount of surfactant(s) and co-surfactant(s) in the aqueous phase is from 0.2 to 0.4;
wherein said disease is selected from the group consisting of: corneal neovascularization disease (NV), ocular melanoma, Gelatinous drop-like corneal dystrophy (GOLD), Secondary corneal amyloidosis—GDLD type, Graves ophthalmopathy, Stevens-Johnson syndrome (SJS), Mooren's ulcer, and Corneal graft rejection.

3. The method according to claim 1, wherein the size distribution of particles in the microemulsion is 15 nm±10 nm.

4. The method according to claim 1, wherein the polydispersity index (POI) of the microemulsion is from 0.02 to 0.380.

5. The method according to claim 1, wherein the Hydrophobic-Lipophilic Balance (HLB) of the surfactants and co-surfactants is from 8 to 16.

6. The method according to claim 1, wherein the oil component is selected from the group consisting of: a natural oil, a synthetic oil, or mixtures thereof.

7. The method according to claim 6, wherein said oil is a vegetable oil or an ester of a medium or a long-chain fatty acid.

8. A method for the treatment of a disease of the anterior segment of the eye comprising the step of using a pharmaceutical composition comprising at least one active ingredient for ophthalmic use in an oil-in-water microemulsion according to claim 1
wherein said disease is selected from the group consisting of: corneal neovascularization disease (NV), ocular melanoma, Gelatinous drop-like corneal dystrophy (GOLD), Secondary corneal amyloidosis—GDLD type, Graves ophthalmopathy, Stevens-Johnson syndrome (SJS), Mooren's ulcer, and Corneal graft rejection.

9. The method according to claim 8, wherein said active ingredient for ophthalmic use is selected from the group consisting of: an anti-angiogenic agent or Multikinase inhibitors agent, immunosuppressant and immunomodulating agent, anti-oxidant agent, and anti-inflammatory agent.

10. The method according to claim 9, wherein said anti-angiogenic agent or Multikinase inhibitors agent is selected from the group consisting of: sorafenib, sorafenib tosylate, regorafenib, regorafenib tosylate, regorafenib isethionate, regorafenib, radotinib, pemigatinib ethylsulfonate and pharmaceutically acceptable derivatives, hydrates, solvates, metabolites and salts thereof, or a polymorphic crystalline form thereof.

11. The method according to claim 9, wherein said immunosuppressant and immunomodulating agent is selected from the group consisting of: interleukin inhibitors and TNF alfa inhibitors.

12. The method according to claim 9, wherein said anti-oxidant agent is selected from the group consisting of:
nordihydroguaiaretic acid, meso-nordihydroguaiaretic (masoprocol) and pharmaceutically acceptable derivatives, hydrates, solvates, metabolites and salts thereof, or a polymorphic crystalline form thereof.

13. The method according to claim 9, wherein said anti-inflammatory agent is selected from the group consisting of: Non-Steroidal Anti-Inflammatory Drugs (NSAIDs) and corticosteroid anti-inflammatory drugs.

14. The method according to claim 8, wherein the pharmaceutical composition comprises from 0.001 mg/ml to 50 mg/ml of said active ingredient for ophthalmic use.

15. The method according to claim 1,
wherein said anterior segment of the eye comprises at least one ocular structure selected from the group consisting of: bulbar conjunctiva, conjunctiva, cornea and sclera.

16. The method according to claim 1,
wherein said anterior segment of the eye is the cornea and said disease is corneal neovascularization disease (NV).
